(19) United States
(12) Patent Application Publication
(10) Pub. No.: US 2021/0345986 A1
(43) Pub. Date: Nov. 11, 2021

(54) AUTOMATIC EVALUATION OF ULTRASOUND PROTOCOL TREES

(71) Applicant: EchoNous, Inc., Redmond, WA (US)

(72) Inventor: Matthew Cook, Woodinville, WA (US)

(21) Appl. No.: 17/068,143

(22) Filed: Oct. 12, 2020

Related U.S. Application Data
(60) Provisional application No. 63/022,987, filed on May 11, 2020.

(51) Int. Cl. A61B 8/08; A61B 8/00; A61B 5/00; A61B 8/14

(52) U.S. Cl. CPC A61B 8/08; A61B 8/463; A61B 8/5246; A61B 5/7267; A61B 8/14; A61B 8/486

(57) ABSTRACT

A facility for performing patient diagnosis is described. The facility accesses a set of diagnostic signs involved in a diagnostic protocol. Until the presence or absence of each of the diagnostic signs of the set has been identified in ultrasound images of a patient, the facility causes an ultrasound image to be captured from the patient, and applies to the captured ultrasound image a trained machine learning model to identify the presence or absence of one or more diagnostic signs of the set. The facility evaluates the diagnostic protocol with respect to the identified presence or absence of each of the set of diagnostic signs to obtain a preliminary diagnosis, and stores the preliminary diagnosis.

### CROSS-REFERENCE TO RELATED APPLICATION

This application claims the benefit of 63/022,987, filed May 11, 2020 and entitled “AUTOMATICALLY IDENTIFYING CLINICALLY IMPORTANT SIGNS IN ULTRASOUND B-MODE AND M-MODE IMAGES FOR PREDICTION OF DIAGNOSES,” which is hereby incorporated by reference in its entirety.

In cases where the present application conflicts with a document incorporated by reference, the present application controls.

### BACKGROUND

Ultrasound imaging is a useful medical imaging modality. For example, internal structures of a patient's body may be imaged before, during or after a therapeutic intervention. A healthcare professional typically holds a portable ultrasound probe, sometimes called a “transducer,” in proximity to the patient and moves the transducer as appropriate to visualize one or more target structures in a region of interest in the patient. A transducer may be placed on the surface of the body or, in some procedures, a transducer is inserted inside the patient's body. The healthcare professional coordinates the movement of the transducer so as to obtain a desired representation on a screen, such as a two-dimensional cross-section of a three-dimensional volume.

Particular views of an organ or other tissue or body feature (such as fluids, bones, joints or the like) can be clinically significant. Such views may be prescribed by clinical standards as views that should be captured by the ultrasound operator, depending on the target organ, diagnostic purpose or the like.

Clinical decision trees have been developed that, given a set of anatomical features (signs) shown in radiological studies such as ultrasound images, return likely diagnoses. Diagnostic protocols based on decision trees are often used by physicians to quickly rule out or confirm specific diagnoses and influence therapeutic decisions.

Among other medical specializations, clinical protocols have been developed for critical care ultrasound imaging of the lungs. For example, the BLUE-protocol (Bedside Lung Ultrasound in Emergency) provides immediate diagnosis of acute respiratory failure, and defines profiles for pneumonia, congestive heart failure, COPD, asthma, pulmonary embolism, and pneumothorax. The FALLS-protocol (Fluid Administration Limited by Lung Sonography) provides decisions for the management of acute circulatory failure, and allows a physician to sequentially rule out obstructive, cardiogenic, hypovolemic, and septic shock. The BLUE- and FALLS-protocols are described in greater detail in the following, each of which is hereby incorporated by reference in its entirety: Lichtenstein DA. BLUE-protocol and FALLS-protocol: two applications of lung ultrasound in the critically ill. Chest. 2015; 147(6):1659-1670. doi:10.1378/chest.14-1313, available at pubmed.ncbi.nlm.nih.gov/26033127; and Lichtenstein, D. A. Lung ultrasound in the critically ill. Ann. Intensive Care 4, 1 (2014), available at doi.org/10.1186/2110-5820-4-1; Tricia Smith, MD; Todd Taylor, MD; Jehangir Meer, MD, Focused Ultrasound for Respiratory Distress: The BLUE Protocol, Emergency Medicine. 2018 January, 50(1):38-40|DOI 10.12788/emed.2018.0077, available at www.mdedge.com/emergencymedicine/article/156882/imaging/emergency-ultrasound-focused-ultrasound-respiratory; and Lichtenstein D. FALLS-protocol: lung ultrasound in hemodynamic assessment of shock, Heart Lung Vessel. 2013; 5(3):142-147, available at www.ncbi.nlm.nih.gov/pmc/articles/PMC3848672.

It is common for diagnostic protocols designed for ultrasound imaging to rely on the capture and evaluation of two different types of ultrasound images: Brightness Mode (“B-Mode”) and Motion Mode (“M-Mode”) images. B-Mode is a two-dimensional ultrasound image composed of bright dots representing ultrasound echoes. The brightness of each dot is determined by the amplitude of the returned echo signal. This allows for visualization and quantification of anatomical structures, as well as for the visualization of diagnostic and therapeutic procedures for small animal studies. In M-Mode ultrasound, pulses are emitted in quick succession—each time, either an A-mode or B-mode image is taken. Over time, this is analogous to recording a video in ultrasound. As the organ boundaries that produce reflections move relative to the probe, this can be used to determine the velocity of specific organ structures. The typical process for capturing an M-Mode image involves manually specifying an M-Mode line with respect to a captured B-Mode image.

### BRIEF DESCRIPTION OF THE DRAWINGS

FIG. 1 is a schematic illustration of a physiological sensing device 10, in accordance with one or more embodiments of the present disclosure.

FIG. 2 is a block diagram showing some of the components typically incorporated in at least some of the computer systems and other devices on which the facility operates.

FIG. 3 is a flow diagram showing a process performed by the facility in some embodiments to make a preliminary diagnosis in accordance with a diagnostic protocol predicated on ultrasound images.

FIG. 4 is a diagnostic protocol drawing showing a sample diagnostic protocol tree used by the facility in some embodiments to diagnose lung disorders.

FIG. 5 is a lung site drawing identifying the lung sites considered by the sample diagnostic protocol tree.

FIG. 6 is a table diagram showing sample contents of a diagnostic signs table in a first state.

FIG. 7 is an ultrasound diagram showing a first sample ultrasound image and the results of detection.

FIG. 8 is a table diagram showing sample contents of the diagnostic signs table in a second state.

FIG. 9 is an ultrasound diagram that shows an example of the facility's determination of an M-Mode line in a B-Mode image.

FIG. 10 is an ultrasound diagram showing a second sample ultrasound image.

FIG. 11 is a table diagram showing sample contents of a diagnostic signs table in a second state.

FIG. 12 is an ultrasound diagram showing a third sample ultrasound image in the result of detection.

FIG. 13 is a table diagram showing sample contents of the diagnostic signs table in a third state.

FIG. 14 is an ultrasound diagram that shows an example of the facility's determination of an M-Mode line in a B-Mode image.

FIG. 15 is an ultrasound diagram showing a fourth sample ultrasound image.

FIG. 16 is a table diagram showing sample contents of a diagnostic signs table in a second state.

FIG. 17 is a diagnostic protocol drawing showing the facility's traversal of the sample diagnostic protocol tree used by the facility in some embodiments to diagnose lung disorders.

FIG. 18 is a display diagram showing a sample display of a preliminary diagnosis by the facility.

FIG. 19 is a model architecture diagram showing an architecture of a neural network used by the facility in some embodiments to recognize signs in ultrasound images.

### DETAILED DESCRIPTION

The inventors have recognized that conventional approaches to using ultrasound to identify all of the necessary signs to make a clinical decision using a decision tree can be time-consuming, as the physician must typically manually examine multiple B-Mode images to assess the presence of certain structures or other signs, and in some cases obtain M-Mode based on features in the B-Mode images. Collecting M-Mode from B-Mode images is particularly burdensome, as the physician must manually specify an M-Mode line in the region of a B-Mode image for which M-Mode is to be captured.

In particular, many lung ultrasound diagnostic protocols rely on determining the presence or absence of ten different signs. Six of these ten signs are identified in B-Mode images: 1) pleural line (bat sign), 2) A-Lines, 3), quad sign 4) tissue sign, 5) fractal/shred sign, and 6) B-lines/lung rockets. The other four of these signs are identified in M-Mode images: 7) seashore sign, 8) sinusoidal sign, 9) stratosphere/barcode sign, and 10) lung point. Conventionally, performing these protocols require the physician to manually identify and keep track of the various signs while switching back and forth between B-Mode and M-Mode on the ultrasound device. The inventors have recognized that this can become burdensome when different regions of the anatomy must be examined, or when the physician must collect different windows of the same region.

In response to recognizing these disadvantages, the inventor has conceived and reduced to practice a software and/or hardware facility that provides automatic assistance for the process of evaluating a diagnostic protocol with respect to ultrasound images (“the facility”). In some embodiments, the facility uses neural networks or machine learning models of other types to automatically identify signs in B-Mode and M-Mode so that the physician does not have to manually search for them.

Further, if M-Mode collection is required to confirm the presence/absence of a particular sign (for example, seashore sign), in some embodiments the facility uses a neural network or a machine learning model of another type to identify the region or regions of a B-Mode image at which the M-Mode line is to be placed. For the four M-mode signs listed above, the M-Mode line must be placed at one or more points across the pleural line. The facility uses object detection or segmentation performed by the neural network to locate the boundaries of the pleural line in the B-Mode image, which enables the facility to automatically place the M-Mode line in the proper location. Once the M-Mode line is identified, in some embodiments the facility automatically collects M-Mode images without requiring user input. The facility then uses a neural network to identify signs in the collected M-mode images.

In some embodiments, once the facility confirms the presence or absence of all of the signs needed by the protocol, it applies the protocol's clinical decision tree to automatically obtain a diagnosis.

In some embodiments, throughout this process, the facility displays the results of sign identification, M-Mode collection, and diagnosis to the user. In some embodiments, this includes displaying text output of sign identification, drawing the M-Mode line on the B-Mode image, and highlighting the returned path of the clinical decision tree.

By operating in some or all of the ways described above, the facility speeds up the process of identifying clinically relevant signs in ultrasound and making clinical diagnoses. Its automatic identification of signs in B-Mode and M-Mode images saves the physician time from manually searching for and keeping track of signs. The facility's automatic placement of M-Mode lines based on the features detected in the B-mode image eases the burden of having to manually select and record M-mode from the ultrasound interface. The facility's evaluation of clinical decision trees given the identified signs provides a faster and more transparent way of suggesting clinical diagnoses. For critical care ultrasound, procedures such as lung evaluation provide urgent diagnoses with immediate therapeutic interventions, so automating the process can lead to significantly more efficient and effective patient care.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by maximizing the usability of ultrasound images by more frequently identifying all structures visualized therein, the facility avoids many cases in which re-imaging is required. By reducing the need to reimage, the facility consumes, overall, less memory and processing resources to capture additional images and perform additional rounds of automatic structure identification. Also, by reducing the amount of time needed to successfully complete a single diagnostic session, the facility permits an organization performing ultrasound imaging to purchase fewer copies of an ultrasound apparatus to serve the same number of patients, or operate an unreduced number of copies at a lower utilization rate, which can extend their useful lifespan, improves their operational status at every time in their lifespan, reduces the need for intra-lifespan servicing and calibration, etc.

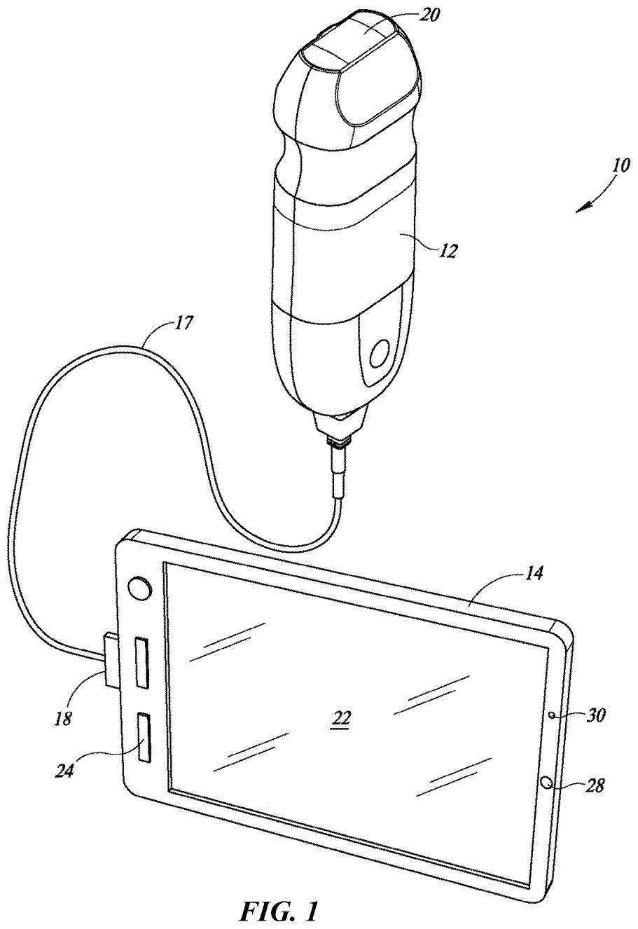

FIG. 1 is a schematic illustration of a physiological sensing device 10, in accordance with one or more embodiments of the present disclosure. The device 10 includes a probe 12 that, in the illustrated embodiment, is electrically coupled to a handheld computing device 14 by a cable 17. The cable 17 includes a connector 18 that detachably connects the probe 12 to the computing device 14. The handheld computing device 14 may be any portable computing device having a display, such as a tablet computer, a smartphone, or the like. In some embodiments, the probe 12 need not be electrically coupled to the handheld computing device 14, but may operate independently of the handheld computing device 14, and the probe 12 may communicate with the handheld computing device 14 via a wireless communication channel.

The probe 12 is configured to transmit an ultrasound signal toward a target structure and to receive echo signals returning from the target structure in response to transmission of the ultrasound signal. The probe 12 includes an ultrasound sensor 20 that, in various embodiments, may include an array of transducer elements (e.g., a transducer array) capable of transmitting an ultrasound signal and receiving subsequent echo signals.

The device 10 further includes processing circuitry and driving circuitry. In part, the processing circuitry controls the transmission of the ultrasound signal from the ultrasound sensor 20. The driving circuitry is operatively coupled to the ultrasound sensor 20 for driving the transmission of the ultrasound signal, e.g., in response to a control signal received from the processing circuitry. The driving circuitry and processor circuitry may be included in one or both of the probe 12 and the handheld computing device 14. The device 10 also includes a power supply that provides power to the driving circuitry for transmission of the ultrasound signal, for example, in a pulsed wave or a continuous wave mode of operation.

The ultrasound sensor 20 of the probe 12 may include one or more transmit transducer elements that transmit the ultrasound signal and one or more receive transducer elements that receive echo signals returning from a target structure in response to transmission of the ultrasound signal. In some embodiments, some or all of the transducer elements of the ultrasound sensor 20 may act as transmit transducer elements during a first period of time and as receive transducer elements during a second period of time that is different than the first period of time (i.e., the same transducer elements may be usable to transmit the ultrasound signal and to receive echo signals at different times).

The computing device 14 shown in FIG. 1 includes a display screen 22 and a user interface 24. The display screen 22 may be a display incorporating any type of display technology including, but not limited to, LCD or LED display technology. The display screen 22 is used to display one or more images generated from echo data obtained from the echo signals received in response to transmission of an ultrasound signal, and in some embodiments, the display screen 22 may be used to display color flow image information, for example, as may be provided in a Color Doppler imaging (CDI) mode. Moreover, in some embodiments, the display screen 22 may be used to display audio waveforms, such as waveforms representative of an acquired or conditioned auscultation signal.

In some embodiments, the display screen 22 may be a touch screen capable of receiving input from a user that touches the screen. In such embodiments, the user interface 24 may include a portion or the entire display screen 22, which is capable of receiving user input via touch. In some embodiments, the user interface 24 may include one or more buttons, knobs, switches, and the like, capable of receiving input from a user of the ultrasound device 10. In some embodiments, the user interface 24 may include a microphone 30 capable of receiving audible input, such as voice commands.

The computing device 14 may further include one or more audio speakers 28 that may be used to output acquired or conditioned auscultation signals, or audible representations of echo signals, blood flow during Doppler ultrasound imaging, or other features derived from operation of the device 10.

The probe 12 includes a housing, which forms an external portion of the probe 12. The housing includes a sensor portion located near a distal end of the housing, and a handle portion located between a proximal end and the distal end of the housing. The handle portion is proximally located with respect to the sensor portion.

The handle portion is a portion of the housing that is gripped by a user to hold, control, and manipulate the probe 12 during use. The handle portion may include gripping features, such as one or more detents, and in some embodiments, the handle portion may have a same general shape as portions of the housing that are distal to, or proximal to, the handle portion.

The housing surrounds internal electronic components and/or circuitry of the probe 12, including, for example, electronics such as driving circuitry, processing circuitry, oscillators, beamforming circuitry, filtering circuitry, and the like. The housing may be formed to surround or at least partially surround externally located portions of the probe 12, such as a sensing surface. The housing may be a sealed housing, such that moisture, liquid or other fluids are prevented from entering the housing. The housing may be formed of any suitable materials, and in some embodiments, the housing is formed of a plastic material. The housing may be formed of a single piece (e.g., a single material that is molded surrounding the internal components) or may be formed of two or more pieces (e.g., upper and lower halves) which are bonded or otherwise attached to one another.

In some embodiments, the probe 12 includes a motion sensor. The motion sensor is operable to sense a motion of the probe 12. The motion sensor is included in or on the probe 12 and may include, for example, one or more accelerometers, magnetometers, or gyroscopes for sensing motion of the probe 12. For example, the motion sensor may be or include any of a piezoelectric, piezoresistive, or capacitive accelerometer capable of sensing motion of the probe 12. In some embodiments, the motion sensor is a tri-axial motion sensor capable of sensing motion about any of three axes. In some embodiments, more than one motion sensor 16 is included in or on the probe 12. In some embodiments, the motion sensor includes at least one accelerometer and at least one gyroscope.

The motion sensor may be housed at least partially within the housing of the probe 12. In some embodiments, the motion sensor is positioned at or near the sensing surface of the probe 12. In some embodiments, the sensing surface is a surface which is operably brought into contact with a patient during an examination, such as for ultrasound imaging or auscultation sensing. The ultrasound sensor 20 and one or more auscultation sensors are positioned on, at, or near the sensing surface.

In some embodiments, the transducer array of the ultrasound sensor 20 is a one-dimensional (1D) array or a two-dimensional (2D) array of transducer elements. The transducer array may include piezoelectric ceramics, such as lead zirconate titanate (PZT), or may be based on microelectromechanical systems (MEMS). For example, in various embodiments, the ultrasound sensor 20 may include piezoelectric micromachined ultrasonic transducers (PMUT), which are microelectromechanical systems (MEMS)-based piezoelectric ultrasonic transducers, or the ultrasound sensor 20 may include capacitive micromachined ultrasound transducers (CMUT) in which the energy transduction is provided due to a change in capacitance.

The ultrasound sensor 20 may further include an ultrasound focusing lens, which may be positioned over the transducer array, and which may form a part of the sensing surface. The focusing lens may be any lens operable to focus a transmitted ultrasound beam from the transducer array toward a patient and/or to focus a reflected ultrasound beam from the patient to the transducer array. The ultrasound focusing lens may have a curved surface shape in some embodiments. The ultrasound focusing lens may have different shapes, depending on a desired application, e.g., a desired operating frequency, or the like. The ultrasound focusing lens may be formed of any suitable material, and in some embodiments, the ultrasound focusing lens is formed of a room-temperature-vulcanizing (RTV) rubber material.

In some embodiments, first and second membranes are positioned adjacent to opposite sides of the ultrasound sensor 20 and form a part of the sensing surface. The membranes may be formed of any suitable material, and in some embodiments, the membranes are formed of a room-temperature-vulcanizing (RTV) rubber material. In some embodiments, the membranes are formed of a same material as the ultrasound focusing lens.

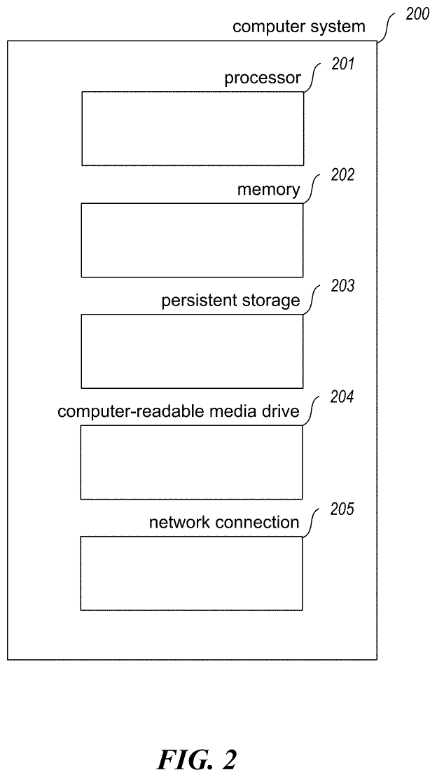

FIG. 2 is a block diagram showing some of the components typically incorporated in at least some of the computer systems and other devices on which the facility operates. In various embodiments, these computer systems and other devices 200 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 201 for executing computer programs and/or training or applying machine learning models, such as a CPU, GPU, TPU, NNP, FPGA, or ASIC; a computer memory 202 for storing programs and data while they are being used, including the facility and associated data, an operating system including a kernel, and device drivers; a persistent storage device 203, such as a hard drive or flash drive for persistently storing programs and data; a computer-readable media drive 204, such as a floppy, CD-ROM, or DVD drive, for reading programs and data stored on a computer-readable medium; and a network connection 205 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

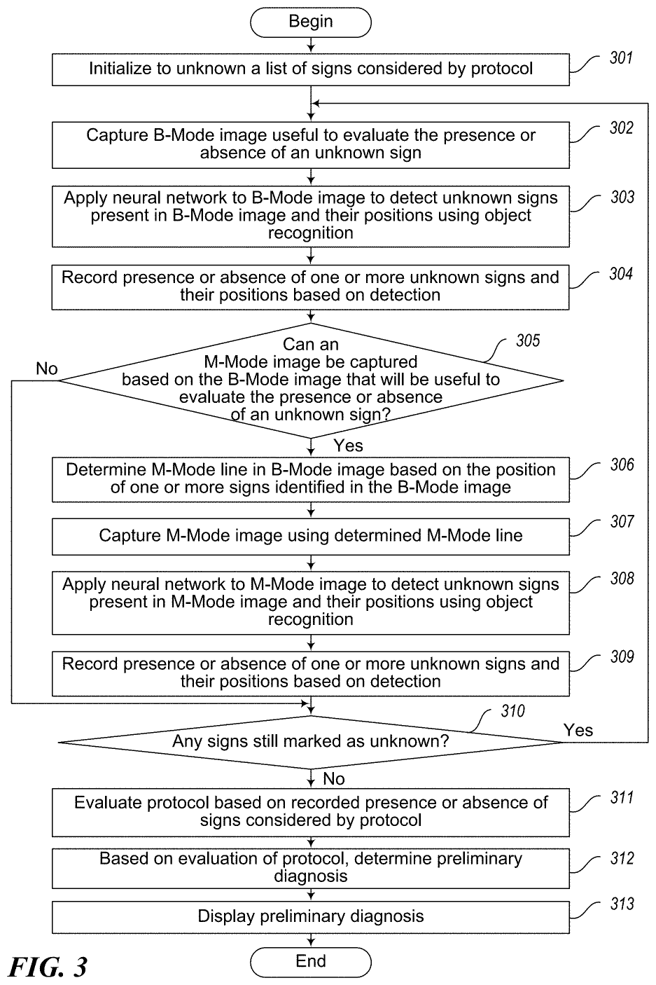

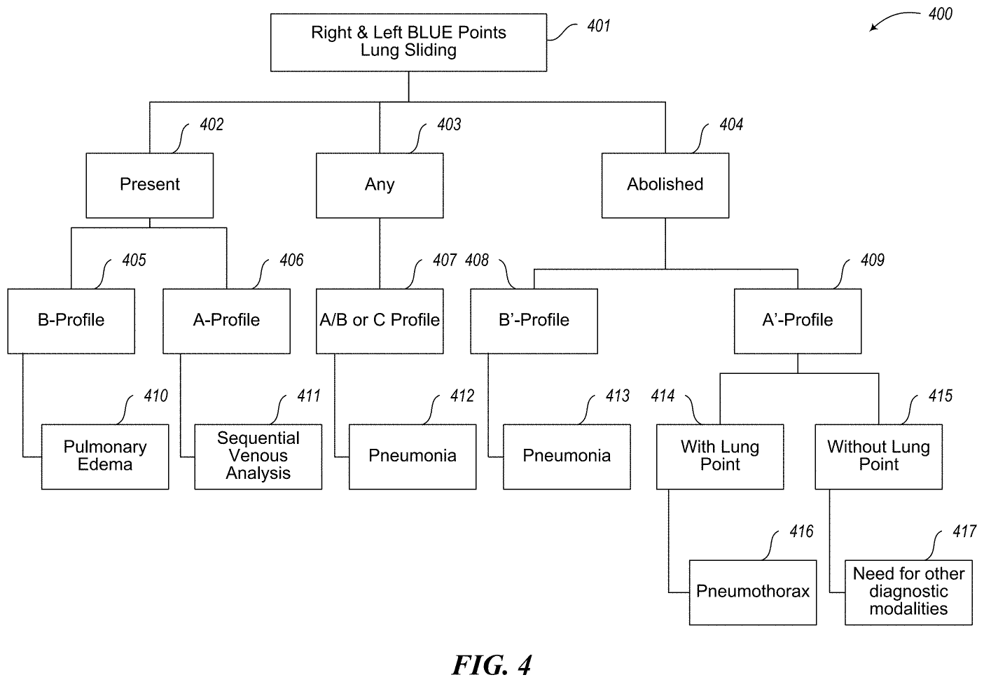

FIG. 3 is a flow diagram showing a process performed by the facility in some embodiments to make a preliminary diagnosis in accordance with a diagnostic protocol predicated on ultrasound images. In act 301, the facility collects a list of diagnostic signs considered by the protocol, and initializes the list to identify all of the signs as initially unknown. Act 301 is discussed in greater detail below in connection with FIGS. 4-6.

FIG. 4 is a diagnostic protocol drawing showing a sample diagnostic protocol tree used by the facility in some embodiments to diagnose lung disorders. The tree 400, corresponding to the BLUE protocol, is comprised of nodes 401-417. In order to reach a diagnosis, the facility proceeds from a root node 401 to one of six leaf nodes 410-413, 416, and 417, each of which specifies a diagnosis. The root node 401 indicates that the protocol considers two different sites in the lung, a right lung BLUE point and a left lung BLUE point; a BLUE point is a site on the front or the back of the body at which an ultrasound transducer is placed in order to obtain an ultrasound image of lung for use in the BLUE protocol.

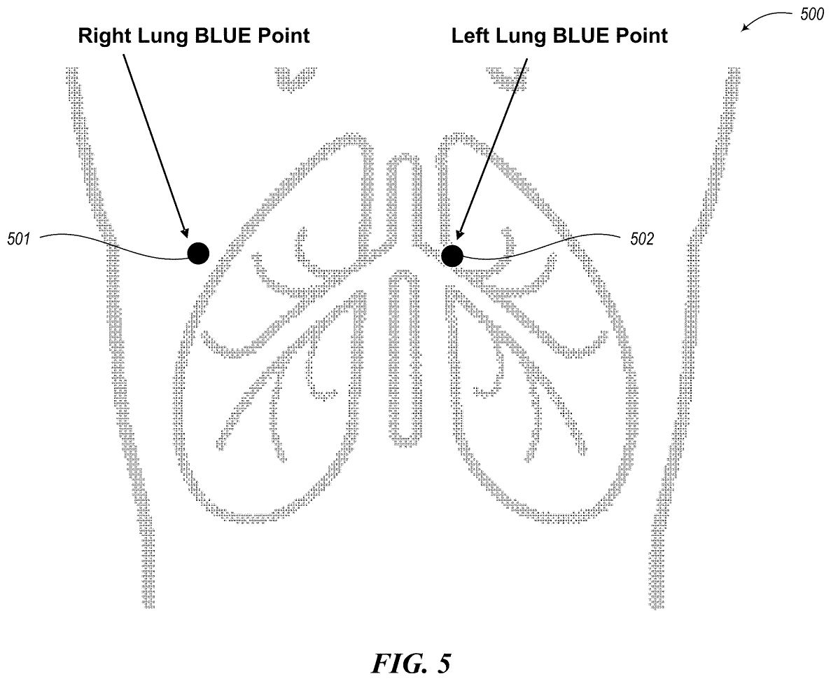

FIG. 5 is a lung site drawing identifying the lung sites considered by the sample diagnostic protocol tree. The diagram 500 identifies the right lung BLUE point 501 and left lung BLUE point 502 relative to the overall shape of the lung 510.

Returning to FIG. 4, the root node 401 also contains an indication that the tree branches on whether a “compound” Lung Sliding sign, that is based on other, “constituent” signs, is present in images of the patient. If the Lung Sliding sign is present, then the facility continues through branch 402; if it is not present, then the facility continues through branch 404; and whether or not the sign is present, the facility is able to continue through branch 403.

At node 402, where the Lung Sliding sign is present, the facility branches based upon whether a B-Profile sign is present or an A-Profile sign is present. If the B-Profile sign is present, then the facility traverses node 405 to leaf node 410 specifying a Pulmonary Edema diagnosis; the B-Profile sign is present if the Lung Sliding sign is present, and at least 3 B-Lines are present in at least one point of each lung. If the A-Profile sign is present, the facility traverses node 406 to leaf node 411 specifying that Sequential Venous Analysis is required for diagnosis; the A-Profile sign is present if the Lung Sliding sign is present, and A-Lines are present and fewer than 3 B-Lines are present at each point of each lung.

At node 404, where the Lung Sliding sign is not present, the facility branches based upon whether a B′-Profile sign is present or an A′-Profile sign is present. If the B′-Profile sign is present, then the facility traverses node 408 to leaf node 413 specifying a Pneumonia diagnosis; the B′-Profile sign is present if the Lung Sliding sign is not present, and at least 3 B-Lines are present in at least one point of each lung. If the A′-Profile sign is present, then the facility traverses node 409; the A′-Profile sign is present if the Lung Sliding sign is not present, and A-Lines are present and fewer than 3 B-Lines are present at each point of each lung. At node 409, if a Lung Point sign is present, then the facility traverses node 414 to leaf node 416, specifying a Pneumothorax diagnosis. At node 409, if a Lung Point sign is not present, then the facility traverses node 415 to leaf node 417, indicating a failure of the protocol to make a diagnosis.

At node 403, where the Lung Sliding sign may or may not be present, if an A/B Profile sign is present or a C Profile sign is present, then the facility proceeds to leaf node 412 specifying a Pneumonia diagnosis; the A/B Profile sign is present if fewer than 3 B-Lines are present in at each point of one lung, and at least 3 B-Lines are present in at least one point of the other lung, while the C profile sign is present if a Tissue Sign or a Fractal/Shred sign is present in at least one point of either lung.

Additional information about the BLUE protocol is as follows: Lung sliding is evaluated through the M-Mode image of an M-Mode line collected across the Pleural Line. The M-Mode signs listed in the table are mutually exclusive; each M-Mode image of an M-Mode line placed across the Pleural Line will be classified as exactly one out of the four possible M-Mode signs listed in the table. The Seashore Sign indicates Lung Sliding. The Stratosphere/Barcode sign indicates no Lung Sliding. The Lung Point is a mix of Seashore Sign and Stratosphere/Barcode Sign, it indicates a point at which part of the lung is sliding, and part of the lung is not sliding (i.e. a collapsed or partially collapsed lung). The Sinusoidal Sign indicates Pleural Effusion, which is orthogonal to the concept of lung sliding, and is not explicitly included in the diagram of the BLUE protocol.

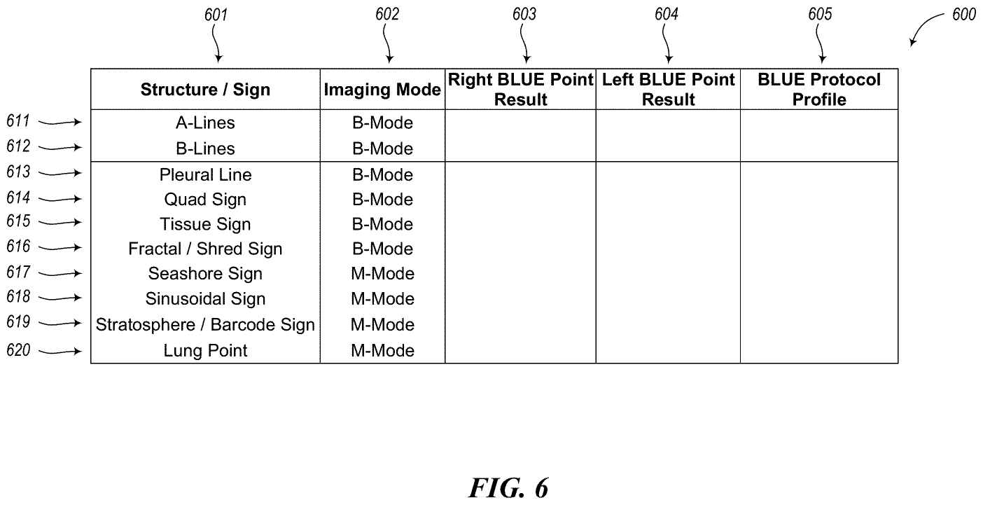

FIG. 6 is a table diagram showing sample contents of a diagnostic signs table in a first state. In particular, the state of table 600 reflects the facility's performance of act 301 to initialize a list of signs considered by the protocol to unknown. The diagnostic signs table is made up of rows, such as rows 611-620, each corresponding to a different sign relied upon by the protocol, in this case the protocol shown in protocol tree 400 shown in FIG. 4. Each row is divided into the following columns: column 601 containing the diagnostic sign to which the row corresponds; column 602 indicating the imaging mode in which the sign can be seen; column 603 indicating whether the sign is present or absent for the patient at the right lung BLUE point site; column 604 indicating whether the sign is present or absent for the patient at the left lung BLUE point site; and column 605 indicating a profile determined in accordance with the diagnostic protocol as a basis for diagnosis. It can be seen that columns 603 and 604 are presently empty, thus identifying all of the signs as unknown for both sites.

While FIG. 6 and each of the table diagrams discussed below show a table whose contents and organization are designed to make them more comprehensible by a human reader, those skilled in the art will appreciate that actual data structures used by the facility to store this information may differ from the table shown, in that they, for example, may be organized in a different manner; may contain more or less information than shown; may be compressed, encrypted, and/or indexed; may contain a much larger number of rows than shown, etc.

Returning to FIG. 3, in act 302, the facility captures a B-Mode image that is useful to evaluate the presence or absence of a sign presently marked as unknown. In various embodiments, the capture of act 302 is fully automatic; performed at the complete discretion of the sonographer; or performed by the sonographer at the direction of the facility. In act 303, the facility applies a neural network or other machine learning model to the B-Mode image captured in act 302 to detect unknown science present in this image and their positions using object recognition.

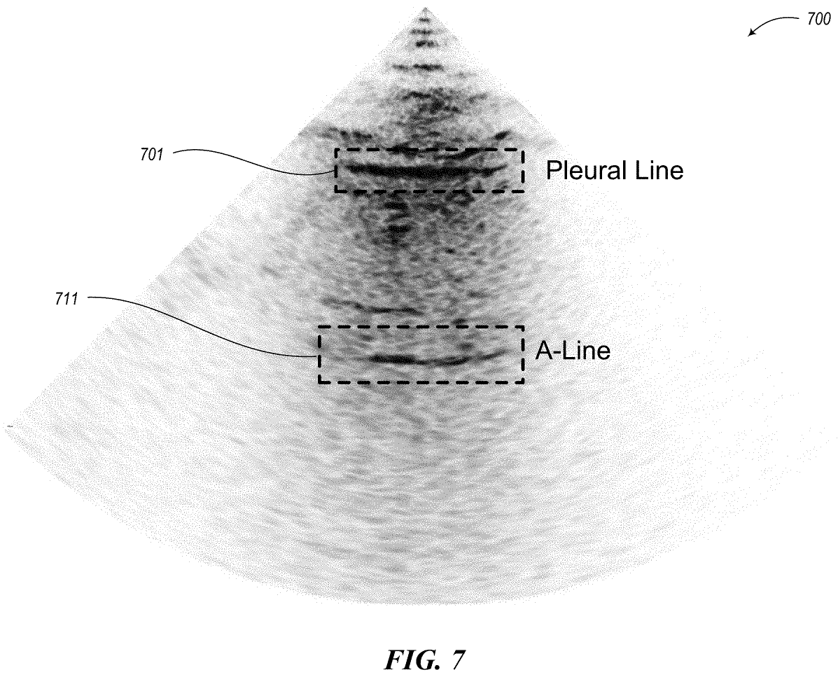

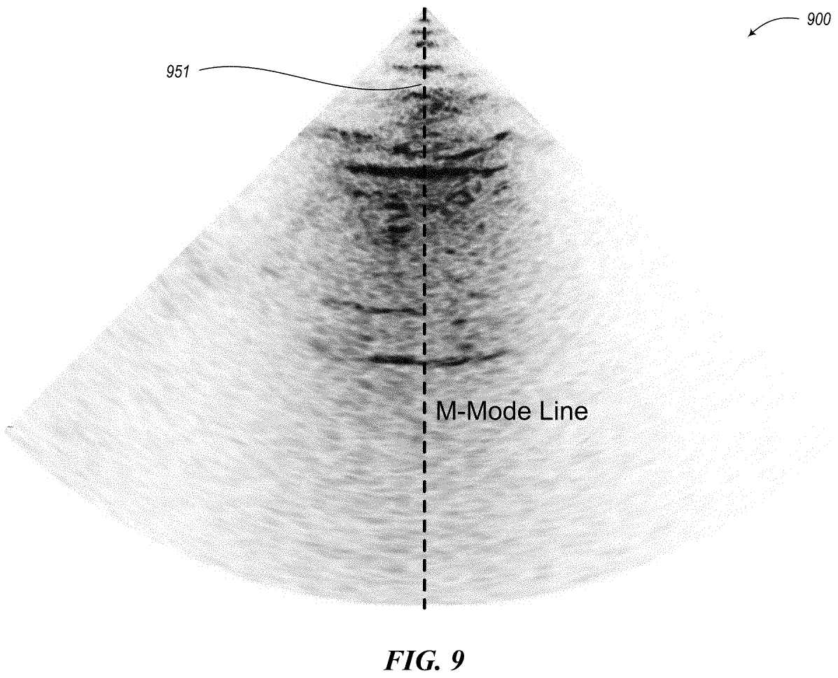

FIG. 7 is an ultrasound diagram showing a first sample ultrasound image and the results of detection. The first sample ultrasound image 700 is a B-Mode image captured on a BLUE point of the front right lung. This sample ultrasound image is grayscale-inverted from its original form in order to be reproducible in a patent drawing at a greater level of fidelity, as are the sample ultrasound images shown in FIGS. 9, 10, 12, 14, and 15 discussed below. It can be seen that the facility detected a Pleural Line 701, and a single A-Line 711.

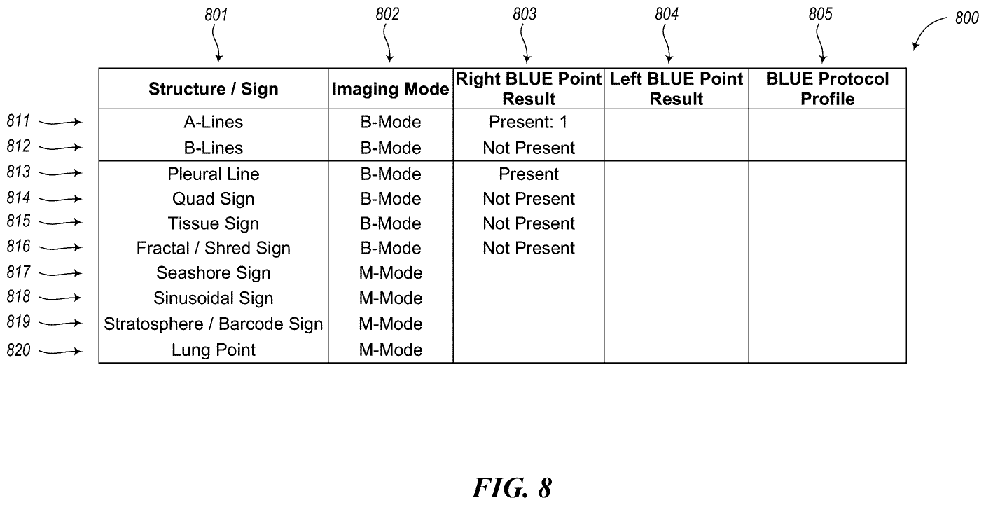

Returning to FIG. 3, in act 304, the facility records the presence or absence of one or more unknown signs and their positions based on the detection performed in act 303. FIG. 8 is a table diagram showing sample contents of the diagnostic signs table in a second state. In particular, the state of table 800 reflects the facility's performance of act 304 with respect to the ultrasound image shown in FIG. 7. It can be seen by the fact that the facility has added the word “present” to the intersection of column 803 with rows 811 and 813 that it has recorded the presence of the signs A-Lines and Pleural Line, both of which were recognized in ultrasound image 700. The numeral 1 at the intersection of column 803 with row 811 indicates that one A-Line was recognized. It can be seen by the fact that the facility has added the phrase “not present” to the intersection of column 803 with rows 812, 814, and 815 that it has recorded the absence of the signs B-Lines, Quad Sign, Tissue Sign, and Fractal/Shred sign, none of which were recognized in ultrasound image 700.

Returning to FIG. 3, in act 305, if an M-Mode image can be captured based on the B-Mode image captured in act 302 that will be useful to evaluate the presence or absence of an unknown sign, then the facility continues in act 306, else the facility continues in act 310. For example, in some embodiments, the facility makes the determination of act 305 based upon whether signs were recognized in the captured B-Mode image that can be used to automatically determine a M-Mode line in the B-Mode image. In act 306, the facility determines an M-Mode line in the B-Mode image captured in act 302 based on the position of one or more signs identified in the B-Mode image. In the case of the ultrasound image 700 shown in FIG. 7, the facility determines an M-Mode line that bisects the Pleural Line 701.

FIG. 9 is an ultrasound diagram that shows an example of the facility's determination of an M-Mode line in a B-Mode image. In particular, it can be seen in image 900 that the facility has determined an M-Mode line 951 that bisects the Pleural Line 701 shown in FIG. 7.

Returning to FIG. 3, in act 307, the facility captures an M-Mode image using the M-Mode line determined in act 306. In various embodiments, the capture of act 307 is fully automatic, or performed with the assistance of the sonographer.

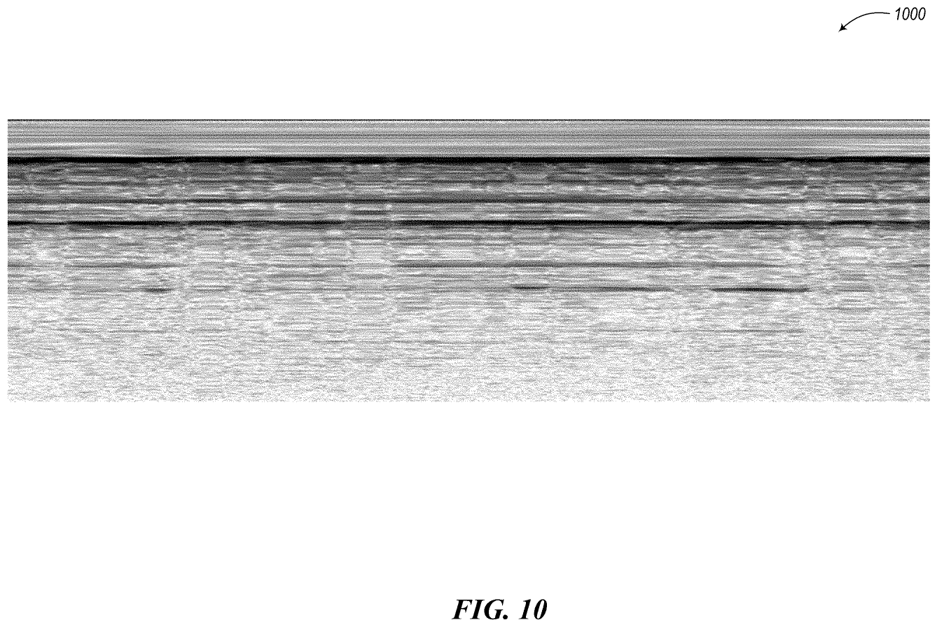

FIG. 10 is an ultrasound diagram showing a second sample ultrasound image. The second sample ultrasound image 1000 is an M-Mode image captured on the upper BLUE point of the front right lung, using the M-Mode line 951 shown in FIG. 9.

Returning to FIG. 3, in act 308, the facility applies a neural network or other machine learning model to detect unknown signs present in the M-Mode image captured in act 307. In some embodiments, the facility trains a first neural network or other machine learning model for recognizing signs in B-Mode images that it applies in act 303, and a separate second neural network or other machine learning model for recognizing signs in M-Mode images that it applies in act 308. In act 309, the facility records the presence or absence of one or more unknown signs in their positions based upon the detection performed in act 308.

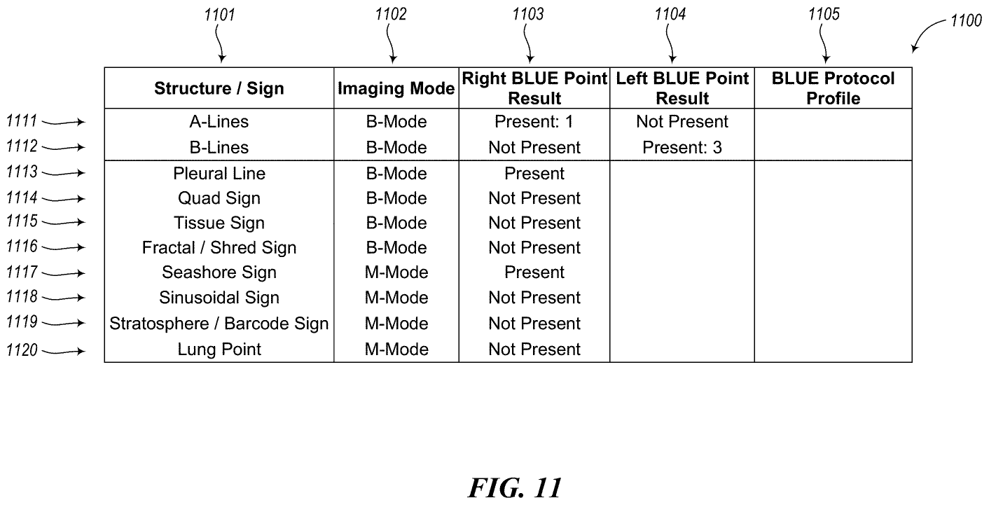

FIG. 11 is a table diagram showing sample contents of a diagnostic signs table in a second state. In particular, the state of table 1100 reflects the facility's performance of act 308 with respect to the ultrasound image shown in FIG. 10. It can be seen by the fact that the facility has added the word “present” to the intersection of column 1103 with row 1117 that it has recorded the presence of the Seashore sign, which was recognized in ultrasound image 1000. It can be seen by the fact that the facility has added the phrase “not present” to the intersection of column 1103 with rows 1118-1120 that it has recorded the absence of the signs Sinusoidal sign, Stratosphere/Barcode Sign, and Lung Point, none of which were recognized in ultrasound image 1000.

Returning to FIG. 3, in act 310, if any signs are still marked as unknown, then the facility continues in act 302 to process additional unknown signs, else the facility continues in act 311. In the example, all of the involved signs remain unknown for the left lung BLUE point imaging site, so the facility continues in act 302. In accordance with act 302 and 303, the facility captures an additional B-Mode image, in which it recognizes additional signs.

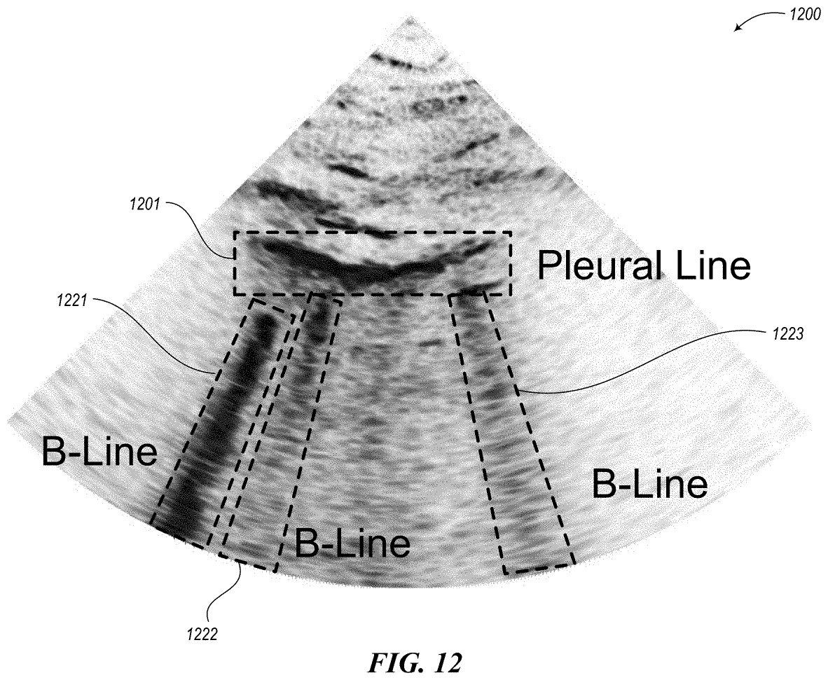

FIG. 12 is an ultrasound diagram showing a third sample ultrasound image in the result of detection. The third sample ultrasound image 1200 is a B-Mode image captured on the lower BLUE point of the front right lung. It can be seen that the facility detected a Pleural Line 1201, and three B-Lines 1221-1223.

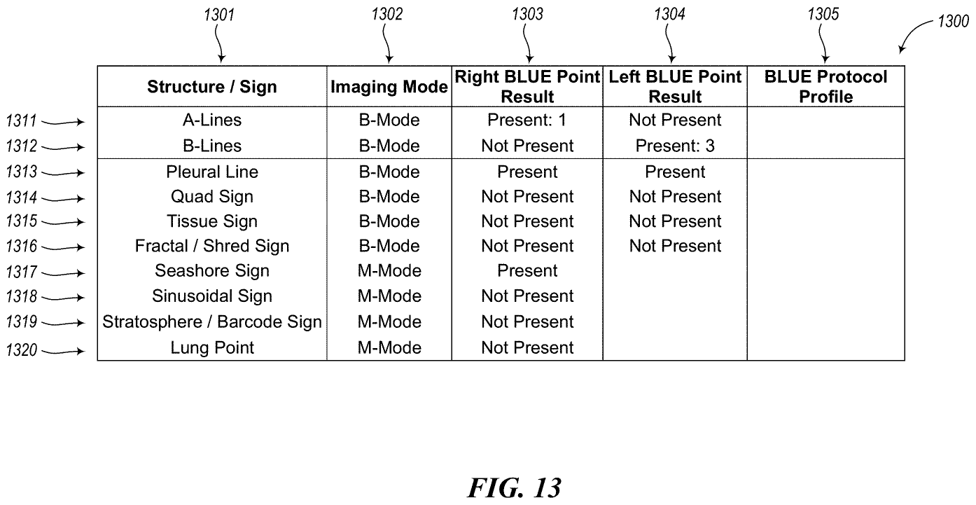

Returning to FIG. 3, in accordance with act 304, the facility records the presence or absence of unknown signs based upon those recognized. FIG. 13 is a table diagram showing sample contents of the diagnostic signs table in a third state. In particular, the state of table 1300 reflects the facility's performance of act 304 with respect to the ultrasound image shown in FIG. 12. It can be seen by the fact that the facility has added the word “present” to the intersection of column 1304 with rows 1312 and 1313 that it has recorded the presence of the signs B-Lines and Pleural Line, both of which were recognized in ultrasound image 1200. The numeral 3 at the intersection of column 1304 with row 1312 indicates that three B-Lines were recognized. It can be seen by the fact that the facility has added the phrase “not present” to the intersection of column 1304 with rows 1311 and 1314-1316 that it has recorded the absence of the signs A-Lines, Quad Sign, Tissue Sign, and Fractal/Shred sign, none of which were recognized in ultrasound image 1200.

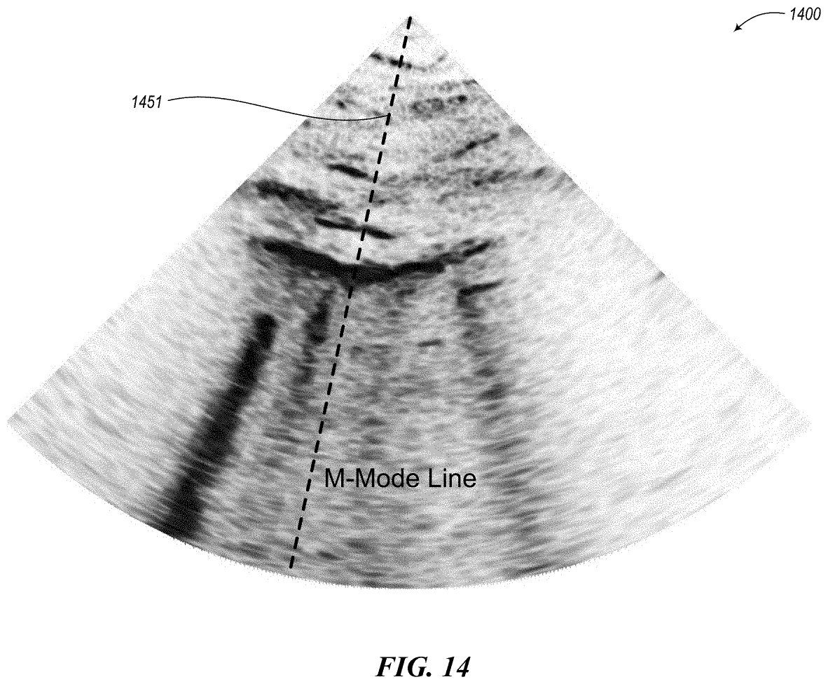

Returning to FIG. 3, in accordance with acts 305-306, the facility determines an M-Mode line in the B-Mode image shown in FIG. 12. FIG. 14 is an ultrasound diagram that shows an example of the facility's determination of an M-Mode line in a B-Mode image. In particular, it can be seen in image 1400 that the facility has determined an M-Mode line 1451 that bisects the Pleural Line 1201 shown in FIG. 12.

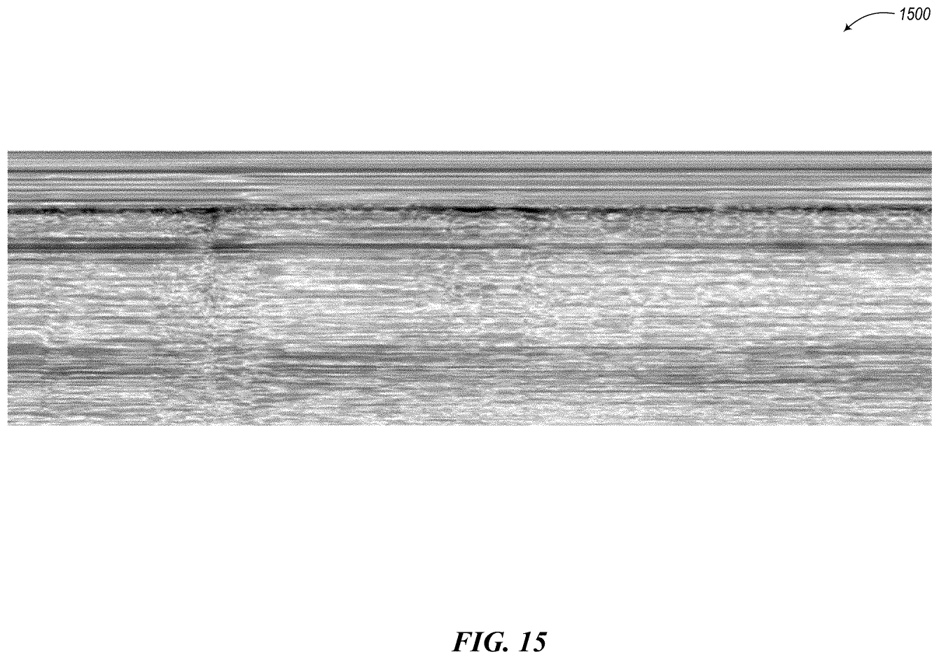

Returning to FIG. 3, in accordance with act 307, the facility captures an M-Mode image using the M-Mode line shown in FIG. 14. FIG. 15 is an ultrasound diagram showing a fourth sample ultrasound image. The fourth sample ultrasound image 1500 is an M-Mode image captured on a BLUE point of the front left lung, using the M-Mode line 1451 shown in FIG. 14.

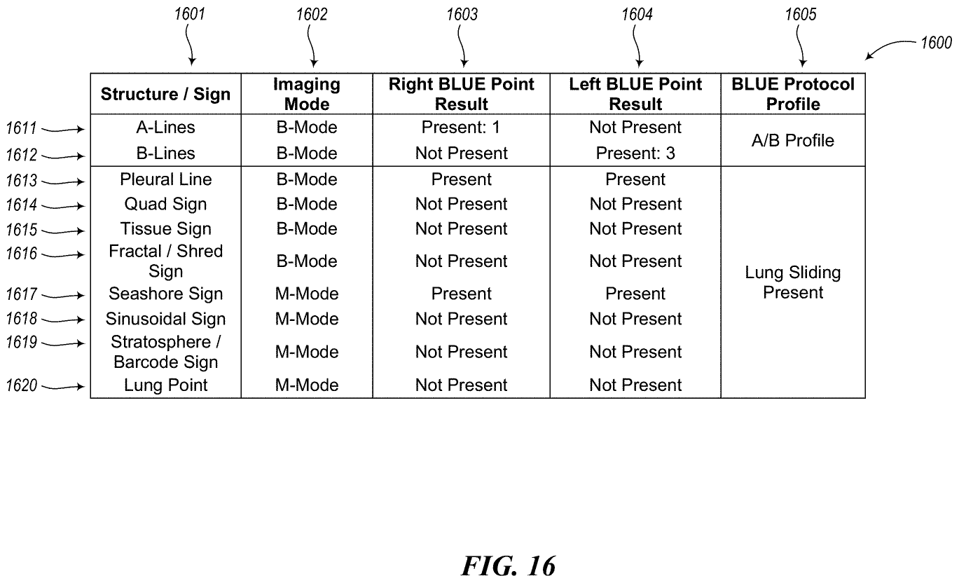

Returning to FIG. 3, in accordance with act 308, the facility detects unknown signs present in the M-Mode image shown in FIG. 15. FIG. 16 is a table diagram showing sample contents of a diagnostic signs table in a second state. In particular, the state of table 1600 reflects the facility's performance of act 308 with respect to the ultrasound image shown in FIG. 15. It can be seen by the fact that the facility has added the word “present” to the intersection of column 1604 with row 1617 that it has recorded the presence of the Seashore sign, which was recognized in ultrasound image 1500. It can be seen by the fact that the facility has added the phrase “not present” to the intersection of column 6004 with rows 1618-1620 that it has recorded the absence of the signs Sinusoidal sign, Stratosphere/Barcode Sign, and Lung Point, none of which were recognized in ultrasound image 1500.

Returning to FIG. 3, in processing the example, in accordance with act 310, the facility determines that no signs are still marked as unknown, and proceeds in act 311. In act 311, the facility evaluates the protocol based on the recorded presence or absence of signs that are considered by the protocol. In a first phase of act 311, the facility determines that the compound signs A/B Profile and Lung Sliding are present based upon the constituent signs on which they depend. Column 1605 in FIG. 16 shows the facility's recording of the presence of these two compound signs. In the second phase of act 311, the facility traverses the protocol tree based upon the presence and absence of involved signs.

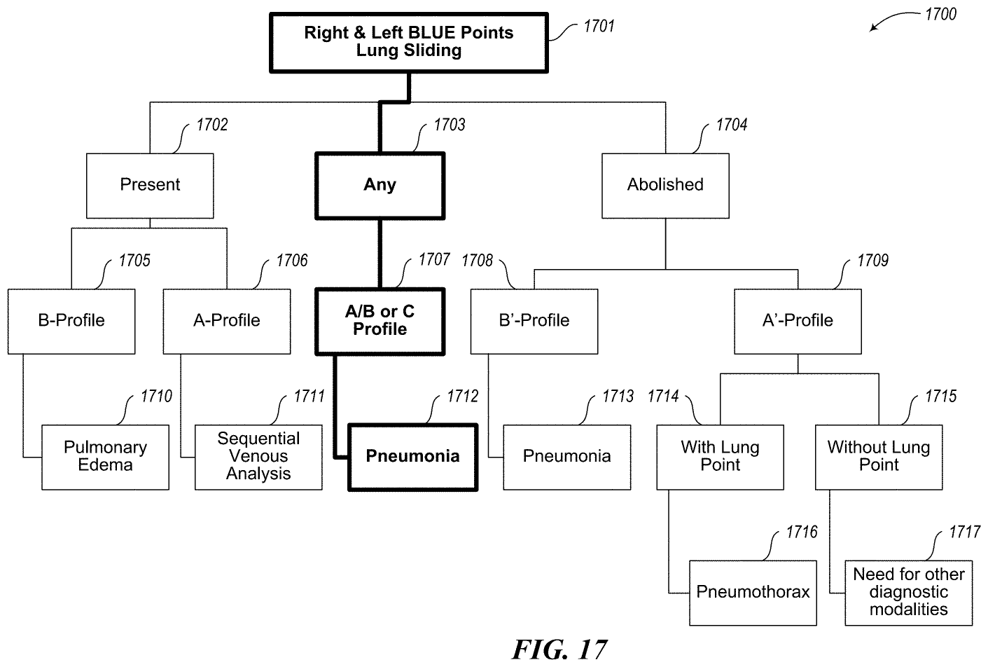

FIG. 17 is a diagnostic protocol drawing showing the facility's traversal of the sample diagnostic protocol tree used by the facility in some embodiments to diagnose lung disorders. In particular, the facility's traversal of the protocol tree 1700 is shown by bold formatting. It can be seen that the facility has traversed branch 1703, which is consistent with the compound sign Lung Sliding being present as it is in the example. The facility further traverses node 1707 based upon the presence of the A/B profile compound sign in the example. In doing so, the facility arrives at leaf node 1712, which specifies a diagnosis of Pneumonia.

Returning to FIG. 3, in act 312, based upon the evaluation of the protocol performed in act 311, the facility determines a preliminary diagnosis. In the case of the example, the facility determines a preliminary diagnosis of Pneumonia, as specified by leaf node 1712 reached in the facility's traversal of the protocol tree. In act 313, the facility displays the preliminary diagnosis determined in act 312. In various embodiments, the facility accomplishes this displaying by altering content displayed on a display device attached or tethered to the ultrasound probe; altering content of a display generated by an app or application running on a smartphone, tablet, laptop computer, desktop computer, etc.; sending it in a message to a physician, nurse, physician's assistant, sonographer, patient, etc., such as an email message, text message, instant message, pager message, etc. After act 313, this process concludes.

Those skilled in the art will appreciate that the acts shown in FIG. 3 may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

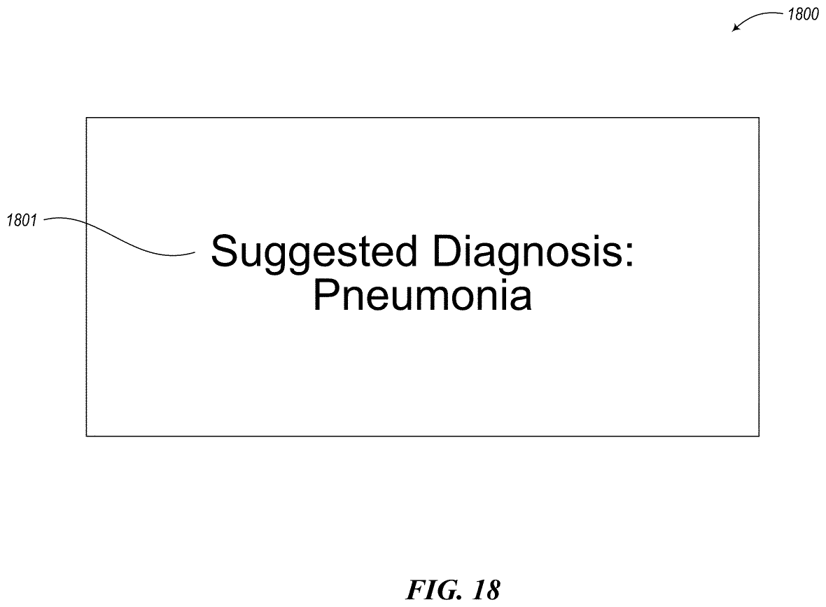

FIG. 18 is a display diagram showing a sample display of a preliminary diagnosis by the facility. In particular, the display 1800 includes text 1801 communicating the preliminary diagnosis.

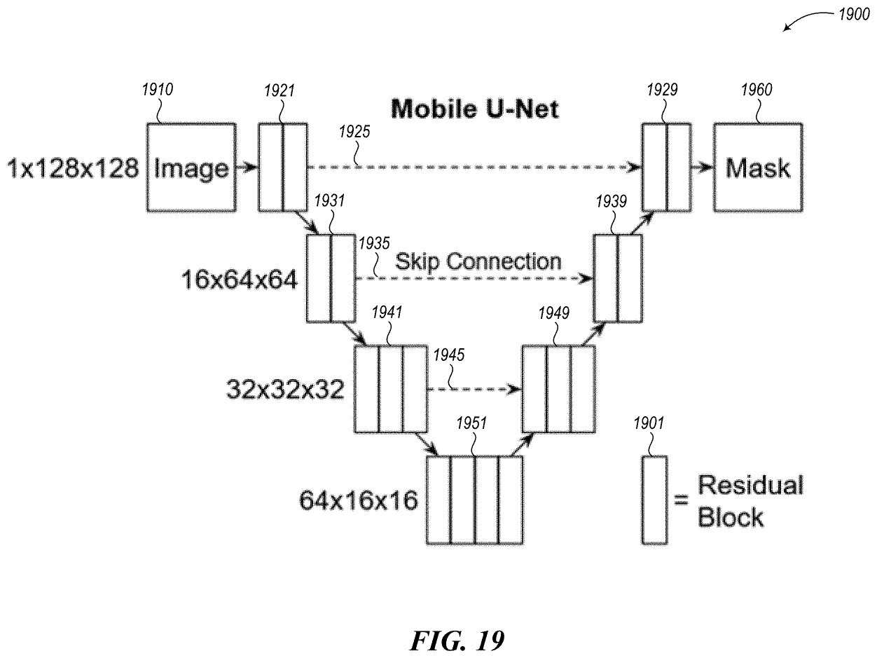

FIG. 19 is a model architecture diagram showing an architecture of a neural network used by the facility in some embodiments to recognize signs in ultrasound images. The diagram shows a Mobile U-Net neural network 1900 used by the facility in some embodiments. In particular, the diagram shows the network's transformation of an image 1910 of pixel dimension 1×128×128 into a mask 1960 of the same dimensions in which recognized structures are identified. The network is comprised of residual blocks 1901. The network makes use of a contracting path through subnetworks 1921, 1931, 1941, 1951, and an expanding path through subnetworks 1951, 1949, 1939, and 1929. During the contraction, spatial information is reduced while feature information is increased. The expansive pathway combines the feature and spatial information through a sequence of up-convolutions and concatenations with high-resolution features from the contracting path received via a skip connections 1925, 1935, 1945. Additional details of Mobile U-Nets are provided by Sanja Scepanovica, Oleg Antropova, Pekka Laurilaa, Vladimir Ignatenkoa, and Jaan Praksc, Wide-Area Land Cover Mapping with Sentinel-1 Imagery using Deep Learning Semantic Segmentation Models, arXiv:1912.05067v2 [eess.IV] 26 Feb. 2020, which is hereby incorporated by reference in its entirety.

In various embodiments, the facility employs various other types of machine learning models to recognize signs. In particular, in various embodiments, the facility uses U-Net neural networks of other types; convolutional neural networks of other types; neural networks of other types; or machine learning models of other types.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system, comprising:
an ultrasound sensing device; and
a computing device, the computing device comprising:
a communication interface configured to directly receive ultrasound echo data sensed by the ultrasound sensing device from a person, the received ultrasound echo data comprising a sequence of ultrasound images;
a memory configured to:
store a representation of a diagnostic protocol tree predicting a diagnosis for a patient based on the presence of a plurality of diagnostic signs in ultrasound images captured from a patient, and
store one or more neural networks trained to identify diagnostic signs among the plurality of diagnostic signs in ultrasound images;
a processor configured to:
apply to each received ultrasound image at least one of the one or more trained neural networks to identify as present or absent in the received ultrasound image diagnostic signs among the plurality of diagnostic signs,
until the plurality of signs are all identified as present or absent in the person, cause the capture of additional ultrasound images from the person by the ultrasound sensing device, and
when all of the plurality of signs are identified as present or absent in the person, use the signs identified as present or absent to evaluate the represented diagnostic protocol tree to obtain a tentative diagnosis of the person; and
a display device configured to:
causing the tentative diagnosis of the person to be displayed.

2. The system of claim 1 wherein the ultrasound sensing device comprises a transducer.

3. The system of 1 wherein the ultrasound images are of a lung of the person.

4. The system of 1 wherein a first ultrasound image of the received sequence of ultrasound images is a B-Mode ultrasound image,
and wherein the processor is further configured to:
in the first ultrasound image, define an M-Mode line relative to at least one diagnostic sign identified as present in the first ultrasound image,
and wherein a second ultrasound image caused to be captured after the defining is an M-Mode ultrasound image captured based on the defined M-Mode line.

5. The system of 4 wherein the trained neural networks stored by the memory comprise a first neural network for identifying diagnostic signs among the plurality of diagnostic signs in B-Mode ultrasound images and a second neural network for identifying diagnostic signs among the plurality of diagnostic signs in M-Mode ultrasound images,
and wherein the first neural network is applied to the first ultrasound image,
and wherein the second neural network is applied to the second ultrasound image.

6. The system of 1 wherein one of the plurality of diagnostic signs is a compound sign that depends on two or more other of the plurality of diagnostic signs,
wherein the processor is further configured to:
determine whether the compound sign is present or absent based on whether the diagnostic signs on which the compound sign depends are identified as present or absent.

7. The system of 1 wherein at least one of the trained neural networks stored by the memory comprises a Mobile U-Net.

8. One or more instances of computer-readable media collectively having contents configured to cause a computing system to perform a method, the method comprising:
accessing a set of diagnostic signs involved in a diagnostic protocol;
until the presence or absence of each of the diagnostic signs of the set has been identified in ultrasound images of a patient:
causing an ultrasound image to be captured from the patient;
applying to the captured ultrasound image a trained machine learning model among one or more trained machine learning models to identify the presence or absence of one or more diagnostic signs of the set;
evaluating the diagnostic protocol with respect to the identified presence or absence of each of the set of diagnostic signs to obtain a preliminary diagnosis; and
storing the preliminary diagnosis.

9. The one or more instances of computer-readable media of claim 8, the method further comprising:
training at least one machine learning model to identify the presence or absence of one or more diagnostic signs of the set.

10. The one or more instances of computer-readable media of claim 8, the method further comprising:
causing the obtained preliminary diagnosis to be displayed.

11. The one or more instances of computer-readable media of claim 8 wherein one of the set of diagnostic signs is a compound sign that depends on two or more other of the set of diagnostic signs,
the method further comprising:
determining whether the compound sign is present or absent based on whether the diagnostic signs on which the compound sign depends are identified as present or absent.

12. The one or more instances of computer-readable media of claim 8 wherein a first captured ultrasound image is a B-Mode ultrasound image,
the method further comprising:
in the first ultrasound image, defining an M-Mode line relative to at least one diagnostic sign identified as present in the first ultrasound image,
and wherein a second ultrasound image caused to be captured after the defining is an M-Mode ultrasound image captured based on the defined M-Mode line.

13. The one or more instances of computer-readable media of claim 12 wherein the one or more trained machine learning models comprise a first machine learning model for identifying diagnostic signs among the set of diagnostic signs in B-Mode ultrasound images and a second machine learning model for identifying diagnostic signs among the set of diagnostic signs in M-Mode ultrasound images, and wherein the first machine learning model is applied to the first ultrasound image, and wherein the second machine learning model is applied to the second ultrasound image.

14. A method in a computing system, the method comprising:
accessing a set of diagnostic signs involved in a diagnostic protocol;
until the presence or absence of each of the diagnostic signs of the set has been identified in ultrasound images of a patient:
causing an ultrasound image to be captured from the patient;
applying to the captured ultrasound image a convolutional neural network among one or more trained convolutional neural networks to identify the presence or absence of one or more diagnostic signs of the set;
evaluating the diagnostic protocol with respect to the identified presence or absence of each of the set of diagnostic signs to obtain a preliminary diagnosis; and
storing the preliminary diagnosis.

15. The method of claim 14, further comprising:
training at least one convolutional neural network to identify the presence or absence of one or more diagnostic signs of the set.

16. The method of claim 14, further comprising:
causing the obtained preliminary diagnosis to be displayed.

17. The method of claim 14 wherein one of the set of diagnostic signs is a compound sign that depends on two or more other of the set of diagnostic signs,
the method further comprising:
determining whether the compound sign is present or absent based on whether the diagnostic signs on which the compound sign depends are identified as present or absent.

18. The method of claim 14 wherein a first captured ultrasound image is a B-Mode ultrasound image,
the method further comprising:
in the first ultrasound image, defining an M-Mode line relative to at least one diagnostic sign identified as present in the first ultrasound image, and wherein a second ultrasound image caused to be captured after the defining is an M-Mode ultrasound image captured based on the defined M-Mode line.

19. The method of claim 18 wherein the one or more trained convolutional neural networks comprise a first convolutional neural network for identifying diagnostic signs among the set of diagnostic signs in B-Mode ultrasound images and a second convolutional neural network for identifying diagnostic signs among the set of diagnostic signs in M-Mode ultrasound images,
and wherein the first convolutional neural network is applied to the first ultrasound image,
and wherein the second convolutional neural network is applied to the second ultrasound image.

20. The system of 19 wherein at least one of the one or more convolutional neural networks comprises a Mobile U-Net.
